(19) United States
(12) Patent Application Publication
(10) Pub. No.: US 2012/0015350 A1
(43) Pub. Date: Jan. 19, 2012

(54) LATERAL FLOW STRIP AND USES THEREOF

(75) Inventors: Arman Nabatiyan, Morton Grove, IL (US); Zaheer Parpia, Evanston, IL (US)

(73) Assignee: NORTHWESTERN UNIVERSITY, Evanston, IL (US)

(21) Appl. No.: 13/255,225

(22) PCT Filed: Mar. 10, 2010
(86) PCT No.: PCT/US10/26802

§ 371 Date: Sep. 28, 2011

Related U.S. Application Data
(60) Provisional application No. 61/158,962, filed on Mar. 10, 2009.

(51) Int. Cl. C12Q 1/70; C12M 1/34; G01N 33/566

(52) U.S. Cl. 435/5; 435/7.1; 436/501; 435/287.2; 422/69; 422/430

(57) ABSTRACT

The present invention relates to lateral flow strip assay system and uses thereof. In particular, the present invention relates to lateral flow assay systems for the simple and inexpensive detection of biomolecules.

### CROSS REFERENCE TO RELATED APPLICATIONS

This application claims priority to U.S. Provisional Patent Application Ser. No. 61/158,962, filed: Mar. 10, 2009, which is herein incorporated by reference in its entirety.

### FIELD OF THE INVENTION

The present invention relates to lateral flow strip assay system and uses thereof. In particular, the present invention relates to lateral flow assay systems for the simple and inexpensive detection of biomolecules.

### BACKGROUND OF THE INVENTION

There is a great need for cost-effective, easy to use systems, methods, and devices for analyzing biological samples. Many commercially available systems cost tens to hundreds of thousands of dollars and have many moving parts which make them prone to failure. Because of the cost and complexity of such systems, their use has generally been limited to clinical laboratories which have the personnel and services needed to support their operation and maintenance.

One class of fully integrated automated analyzers, represented by the Abbott Architect, Siemens Centaur, Roche Elecsys, and others, perform immunoassays. Another class of modular analyzers, represented by the Abbott m2000, Roche COBAS, bioMérieux NucliSENS and others, perform nucleic acid assays. Much of the complexity of these systems is a result of separation steps involved in processing the assays.

Modular systems are also frequently used in research laboratories. Immunoassay separations may be performed by plate washers such as Titertek MAP-C2, BioTek ELx50, Tecan PW 96/384 and others. Nucleic acid separations are performed by systems such as the Applied Biosystems PRISM™ 6100, Invitrogen iPrep, Thermo Scientific KingFisher, Promega Maxwell, and others.

The availability of low-cost, reliable analyzers is of particular concern as it relates to the diagnosis and management of disease around the world. This problem is vividly illustrated by the problems associated with management of HIV infections. Many technologies exist that permit detection of nucleic acids or protein levels associated with HIV. This detection is important for managing the patient care of those infected by HIV. However, the cost and complexity of these systems prohibits their widespread use.

Existing assay systems and methods are complex, expensive and not suitable for use in many settings, especially in the developing world. Additional systems and methods are needed.

### SUMMARY OF THE INVENTION

The present invention relates to lateral flow strip assay system and uses thereof. In particular, the present invention relates to lateral flow assay systems for the simple and inexpensive detection of biomolecules.

Embodiments of the present invention provide an assay device and kits and systems comprising said assay device for use in the detection of the presence or absence of an analyte in a sample, comprising: (a) A sample receiving membrane which conducts flow of a sample and is in flow contact with: (b) An analyte detection membrane which conducts flow of the sample, comprising one or more of i) a labeling reagent absorption zone comprising a labeling reagent, ii) an analyte-reagent complex capture zone comprising an analyte capture reagent, iii) a control reagent capture zone comprising control reagent; and a sacrificial zone comprising non-specific binders (e.g., immunoglobulins), wherein the labeling reagent is capable of forming a complex with an analyte to form an analyte-labeling reagent complex, the analyte capture reagent is capable of binding the analyte-labeling reagent complex, the control reagent is capable of binding the labeling reagent, and the non-specific binders bind to unbound analyte specific antibodies or other analyte specific components in the sample. In some embodiments, the flow contact between the sample receiving membrane and analyte detection membrane is lateral flow contact. In some embodiments, the labeling reagent comprises an antibody specific for the analyte (e.g., an IgG antibody, an IgM antibody, an IgA antibody or a portion thereof). In some embodiments, the antibody or portion thereof is derived from mouse, goat, sheep, rat, rabbit, cow, human or chimeras thereof. In some embodiments, the sample receiving membrane and the analyte detection membrane are enclosed in a housing. In some embodiments, the device comprises an absorbent sink in lateral flow contact with the analyte detection membrane. In some embodiments, the housing comprises a sample application aperture and an observation window positioned to display the labeling reagent capture zone, a detection zone and the control zone. In some embodiments, a backing is laminated or otherwise affixed to the bottom surface of the sample receiving membrane and the analyte detection membrane. In some embodiments, this laminate compromises a semi-rigid material of at least 0.005 inches thick. In some embodiments, the sample receiving membrane, analyte detection membrane and absorbent sink are enclosed in a housing comprising a sample application aperture and an observation window positioned to display the labeling reagent absorption zone, the detection capture zone and control zone. In some embodiments, the sacrificial zone is located approximately 14 mm from the distal end of the sample receiving membrane and the analyte-reagent complex capture zone is located approximately 16 mm from the distal end of the sample receiving membrane. In some embodiments, the analyte capture reagent comprises a label (e.g., fluorescent or other label). In some embodiments, the label is contained in a microsphere.

Additional embodiments of the present invention provide a method of detecting the presence of an analyte in a sample comprising: applying a sample to an assay device as described herein, wherein the sample flows from the sample receiving membrane to the analyte detection membrane under conditions such that the labeling reagent forms a complex with the analyte to form an analyte-labeling reagent complex and the analyte capture reagent binds to the analyte-labeling reagent complex; and detecting the presence of the analyte. In some embodiments, the analyte includes, but is not limited to a protein, peptide, small molecule; antibody, nucleic acid, virus, virus particle, drug, drug metabolite or small molecule. Specific examples include, but are not limited to, human chorionic gonadotrophin, luteinizing hormone, estrone-3-glucoronide, pregnanedio13-glucoronide, insulin, glucagon, relaxin, thyrotropin, somatotropin, gonadotropin, follicle-stimulating hormone, gastrin, bradykinin, vasopressin, polysaccharides, estrone, estradiol, cortisol, testosterone, progesterone, chenodeoxycholic acid, digoxin, cholic acid, digitoxin, deoxycholic acid, lithocholic acids; vitamins, thyroxine, triiodothyronine, histamine, serotorin, prostaglandin, drugs, drug metabolites, ferritin or CEA. Exemplary sample types include, but are not limited to, blood, serum, nasal fluid, urine, sweat, plasma, semen, cerebrospinal fluid, tears, pus, amniotic fluid, saliva, lung aspirate, gastrointestinal contents, vaginal discharge, urethral discharge, chorionic villi specimens, skin epithelials, genitalia epithelials, gum epithelials, throat epithelials, hair or sputum, as well as environmental samples.

Additional embodiments are described herein.

### DESCRIPTION OF THE FIGURES

FIG. 1 shows a diagram of a lateral flow assay used in embodiments of the present invention.

FIG. 2 shows a diagram of an optimized lateral flow assay used in embodiments of the present invention.

FIG. 3 shows an epifluorescent image of test strips with p24 antigen.

FIG. 4 shows a diagram of an exemplary lateral flow assay of embodiments of the present invention.

FIG. 5 shows an epifluorescent image of test strips with p24 antigen.

FIG. 6 shows a graph of the effect of bead binding to a sacrificial line.

FIG. 7 shows a graph of a dose response of p24 antigen for test strips with or without a sacrificial test line.

FIG. 8 shows a graph of a dose response of p24 antigen for test strips with a sacrificial test line.

FIG. 9 shows a graph of a dose response of p24 antigen for test strips with a sacrificial mouse antibody present in the reaction or on a test line.

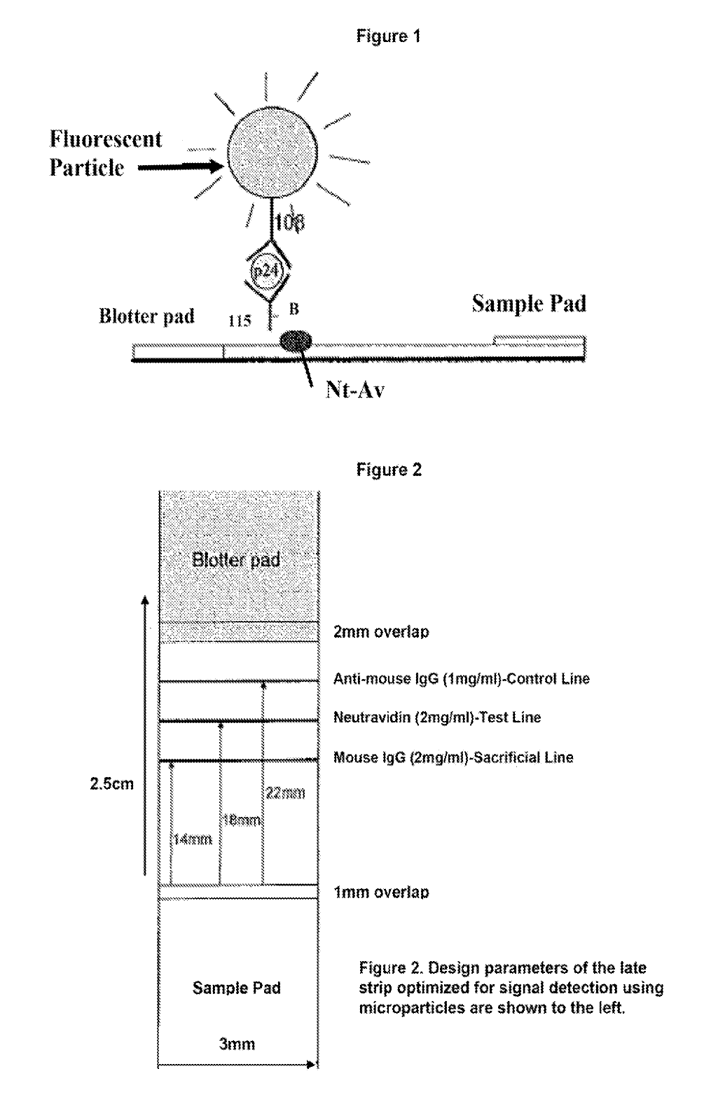

### DEFINITIONS

To facilitate an understanding of this disclosure, terms are defined below:

“Purified polypeptide” or “purified protein” or “purified nucleic acid” means a polypeptide or nucleic acid of interest or fragment thereof which is essentially free of, e.g., contains less than about 50%, preferably less than about 70%, and more preferably less than about 90%, cellular components with which the polypeptide or polynucleotide of interest is naturally associated.

The term “isolated” means that the material is removed from its original environment (e.g., the natural environment if it is naturally occurring). For example, a naturally-occurring polynucleotide or polypeptide present in a living animal is not isolated, but the same polynucleotide or DNA or polypeptide, which is separated from some or all of the coexisting materials in the natural system, is isolated. Such polynucleotide could be part of a vector and/or such polynucleotide or polypeptide could be part of a composition, and still be isolated in that the vector or composition is not part of its natural environment.

“Polypeptide” and “protein” are used interchangeably herein and include all polypeptides as described below. The basic structure of polypeptides is well known and has been described in innumerable textbooks and other publications in the art. In this context, the term is used herein to refer to any peptide or protein comprising two or more amino acids joined to each other in a linear chain by peptide bonds. As used herein, the term refers to both short chains, which also commonly are referred to in the art as peptides, oligopeptides and oligomers, for example, and to longer chains, which generally are referred to in the art as proteins, of which there are many types.

It will be appreciated that polypeptides often contain amino acids other than the 20 amino acids commonly referred to as the 20 naturally occurring amino acids, and that many amino acids, including the terminal amino acids, may be modified in a given polypeptide, either by natural processes, such as processing and other post-translational modifications, but also by chemical modification techniques which are well known to the art. Even the common modifications that occur naturally in polypeptides are too numerous to list exhaustively here, but they are well described in basic texts and in more detailed monographs, as well as in a voluminous research literature, and they are well known to those of skill in the art. Among the known modifications which may be present in polypeptides of the present are, to name an illustrative few, acetylation, acylation, ADP-ribosylation, amidation, covalent attachment of flavin, covalent attachment of a heme moiety, covalent attachment of a nucleotide or nucleotide derivative, covalent attachment of a lipid of lipid derivative, covalent attachment of phosphatidylinositol, cross-linking, cyclization, disulfide bond formation, demethylation, formation of covalent cross-links, formation of cystine, formation of pyroglutamate, formylation, gamma-carboxylation, glycosylation, GPI anchor formation, hydroxylation, iodination, methylation, myrisoylation, oxidation, proteolytic processing, phosphorylation, prenylation, racemization, selenoylation, sulfation, transfer-RNA mediated addition of amino acids to proteins such as arginylation, and ubiquitination.

Such modifications are well known to those of skill and have been described in great detail in the scientific literature. Several particularly common modifications, glycosylation, lipid attachment, sulfation, gamma-carboxylation of glutamic acid residues, hydroxylation and ADP-ribosylation, for instance, are described in most basic texts, such as for instance Proteins—Structure and Molecular Properties, 2.sup.nd Ed., T. E. Creighton, W. H. Freeman and Company, New York (1993). Many detailed reviews are available on this subject, such as, for example, those provided by Wold, F., Posttranslational Protein Modifications: Perspectives and Prospects, pg. 1-12 in Posttranslational Covalent Modification of Proteins, B. C. Johnson, Ed., Academic Press, New York (1983); Seifter et al., Analysis for protein modifications and nonprotein cofactors, Meth. Enzymol. 182: 626-646 (1990) and Rattan et al., Protein synthesis: Posttranslational Modifications and Aging, Ann N.Y. Acad. Sci. 663: 48-62 (1992). It will be appreciated, as is well known and as noted above, that polypeptides are not always entirely linear. For instance, polypeptides may be branched as a result of ubiquitination, and they may be circular, with or without branching, generally as a result of posttranslational events, including natural processing events and events brought about by human manipulation which do not occur naturally. Circular, branched, and branched circular polypeptides may be synthesized by non-translational natural process and by entirely synthetic methods as well. Modifications can occur anywhere in a polypeptide, including the peptide backbone, the amino acid side-chains and the amino or carboxyl termini. In fact, blockage of the amino or carboxyl group in a polypeptide, or both, by a covalent modification, is common in naturally occurring and synthetic polypeptides. For instance, the amino terminal residue of polypeptides made in E. coli, prior to proteolytic processing, almost invariably will be N-formylmethionine.

The modifications that occur in a polypeptide often will be a function of how it is made. For polypeptides made by expressing a cloned gene in a host, for instance, the nature and extent of the modifications in large part will be determined by the host cell posttranslational modification capacity and the modification signals present in the polypeptide amino acid sequence. For instance, as is well known, glycosylation often does not occur in bacterial hosts such as E. coli. Accordingly, when glycosylation is desired, a polypeptide should be expressed in a glycosylating host, generally a eukaryotic cell. Insect cells often carry out the same posttranslational glycosylations as mammalian cells, and, for this reason, insect cell expression systems have been developed to express efficiently mammalian proteins having native patterns of glycosylation. Similar considerations apply to other modifications.

It will be appreciated that the same type of modification may be present in the same or varying degree at several sites in a given polypeptide. Also, a given polypeptide may contain many types of modifications.

In general, as used herein, the term polypeptide encompasses all such modifications, particularly those that are present in polypeptides synthesized by expressing a polynucleotide in a host cell.

The term “mature” polypeptide refers to a polypeptide which has undergone a complete, post-translational modification appropriate for the subject polypeptide and the cell of origin.

A “fragment” of a specified polypeptide refers to an amino acid sequence which comprises at least about 3-5 amino acids, more preferably at least about 8-10 amino acids, and even more preferably at least about 15-20 amino acids derived from the specified polypeptide. The term “immunologically identifiable with/as” refers to the presence of epitope(s) and polypeptide(s) which also are present in and are unique to the designated polypeptide(s). Immunological identity may be determined by antibody binding and/or competition in binding. The uniqueness of an epitope also can be determined by computer searches of known data banks, such as GenBank, for the polynucleotide sequence which encodes the epitope and by amino acid sequence comparisons with other known proteins.

As used herein, “epitope” means an antigenic determinant of a polypeptide or protein. Conceivably, an epitope can comprise three amino acids in a spatial conformation which is unique to the epitope. Generally, an epitope consists of at least five such amino acids and more usually, it consists of at least eight to ten amino acids. Methods of examining spatial conformation are known in the art and include, for example, x-ray crystallography and two-dimensional nuclear magnetic resonance.

A “conformational epitope” is an epitope that is comprised of a specific juxtaposition of amino acids in an immunologically recognizable structure, such amino acids being present on the same polypeptide in a contiguous or non-contiguous order or present on different polypeptides. A polypeptide is “immunologically reactive” with an antibody when it binds to an antibody due to antibody recognition of a specific epitope contained within the polypeptide. Immunological reactivity may be determined by antibody binding, more particularly, by the kinetics of antibody binding, and/or by competition in binding using as competitor(s) a known polypeptide(s) containing an epitope against which the antibody is directed. The methods for determining whether a polypeptide is immunologically reactive with an antibody are known in the art.

As used herein, the term “immunogenic polypeptide containing an epitope of interest” means naturally occurring polypeptides of interest or fragments thereof, as well as polypeptides prepared by other means, for example, by chemical synthesis or the expression of the polypeptide in a recombinant organism.

“Purified product” refers to a preparation of the product which has been isolated from the cellular constituents with which the product is normally associated and from other types of cells which may be present in the sample of interest.

“Analyte,” as used herein, is the substance to be detected which may be present in the test sample, including, biological molecules of interest, small molecules, pathogens, and the like. The analyte can include a protein, a polypeptide, an amino acid, a nucleotide target and the like. The analyte can be soluble in a body fluid such as blood, blood plasma or serum, urine or the like. The analyte can be in a tissue, either on a cell surface or within a cell. The analyte can be on or in a cell dispersed in a body fluid such as blood, urine, breast aspirate, or obtained as a biopsy sample.

A “specific binding member,” as used herein, is a member of a specific binding pair. That is, two different molecules where one of the molecules, through chemical or physical means, specifically binds to the second molecule. Therefore, in addition to antigen and antibody specific binding pairs of common immunoassays, other specific binding pairs can include biotin and avidin, carbohydrates and lectins, complementary nucleotide sequences, effector and receptor molecules, cofactors and enzymes, enzyme inhibitors, and enzymes and the like. Furthermore, specific binding pairs can include members that are analogs of the original specific binding members, for example, an analyte-analog. Immunoreactive specific binding members include antigens, antigen fragments, antibodies and antibody fragments, both monoclonal and polyclonal and complexes thereof, including those formed by recombinant DNA molecules.

Specific binding members include “specific binding molecules.” A “specific binding molecule” intends any specific binding member, particularly an immunoreactive specific binding member. As such, the term “specific binding molecule” encompasses antibody molecules (obtained from both polyclonal and monoclonal preparations), as well as, the following: hybrid (chimeric) antibody molecules (see, for example, Winter, et al., Nature 349: 293-299 (1991), and U.S. Pat. No. 4,816,567); F(ab′).sub.2 and F(ab) fragments; Fv molecules (non-covalent heterodimers, see, for example, Inbar, et al., Proc. Natl. Acad. Sci. USA 69: 2659-2662 (1972), and Ehrlich, et al., Biochem. 19: 4091-4096 (1980)); single chain Fv molecules (sFv) (see, for example, Huston, et al., Proc. Natl. Acad. Sci. USA 85: 5879-5883 (1988)); humanized antibody molecules (see, for example, Riechmann, et al., Nature 332: 323-327 (1988), Verhoeyan, et al., Science 239: 1534-1536 (1988), and UK Patent Publication No. GB 2,276,169, published 21 Sep. 1994); and, any functional fragments obtained from such molecules, wherein such fragments retain immunological binding properties of the parent antibody molecule.

The term “hapten,” as used herein, refers to a partial antigen or non-protein binding member which is capable of binding to an antibody, but which is not capable of eliciting antibody formation unless coupled to a carrier protein.

A “capture reagent,” as used herein, refers to an unlabeled specific binding member which is specific either for the analyte as in a sandwich assay, for the indicator reagent or analyte as in a competitive assay, or for an ancillary specific binding member, which itself is specific for the analyte, as in an indirect assay. The capture reagent can be directly or indirectly bound to a solid phase material before the performance of the assay or during the performance of the assay, thereby enabling the separation of immobilized complexes from the test sample.

The “indicator reagent” comprises a “signal-generating compound” (“label”) which is capable of generating and generates a measurable signal detectable by external means. In some embodiments, the indicator reagent is conjugated (“attached”) to a specific binding member. In addition to being an antibody member of a specific binding pair, the indicator reagent also can be a member of any specific binding pair, including either hapten-anti-hapten systems such as biotin or anti-biotin, avidin or biotin, a carbohydrate or a lectin, a complementary nucleotide sequence, an effector or a receptor molecule, an enzyme cofactor and an enzyme, an enzyme inhibitor or an enzyme and the like. An immunoreactive specific binding member can be an antibody, an antigen, or an antibody/antigen complex that is capable of binding either to the polypeptide of interest as in a sandwich assay, to the capture reagent as in a competitive assay, or to the ancillary specific binding member as in an indirect assay. When describing probes and probe assays, the term “reporter molecule” may be used. A reporter molecule comprises a signal generating compound as described hereinabove conjugated to a specific binding member of a specific binding pair, such as carbazole or adamantane.

The various “signal-generating compounds” (labels) contemplated include chromagens, catalysts such as enzymes, luminescent compounds such as fluorescein and rhodamine, chemiluminescent compounds such as dioxetanes, acridiniums, phenanthridiniums and luminol, radioactive elements and direct visual labels. Examples of enzymes include alkaline phosphatase, horseradish peroxidase, beta-galactosidase and the like. The selection of a particular label is not critical, but it should be capable of producing a signal either by itself or in conjunction with one or more additional substances.

“Solid phases” (“solid supports”) are known to those in the art and include the walls of wells of a reaction tray, test tubes, polystyrene beads, magnetic or non-magnetic beads, nitrocellulose strips or other lateral flow strips, membranes, microparticles such as latex particles, and others. The “solid phase” is not critical and can be selected by one skilled in the art. Thus, latex particles, microparticles, magnetic or non-magnetic beads, membranes, plastic tubes, walls of microtiter wells, glass or silicon chips, are all suitable examples. It is contemplated and within the scope of the present invention that the solid phase also can comprise any suitable porous material.

As used herein, the terms “detect”, “detecting”, or “detection” may describe either the general act of discovering or discerning or the specific observation of a detectably labeled composition.

The term “polynucleotide” refers to a polymer of ribonucleic acid (RNA), deoxyribonucleic acid (DNA), modified RNA or DNA, or RNA or DNA mimetics. This term, therefore, includes polynucleotides composed of naturally-occurring nucleobases, sugars and covalent internucleoside (backbone) linkages as well as polynucleotides having non-naturally-occurring portions which function similarly. Such modified or substituted polynucleotides are well-known in the art and for the purposes of the present invention, are referred to as “analogues.”

As used herein, the term “nucleic acid molecule” refers to any nucleic acid containing molecule, including but not limited to, DNA or RNA. The term encompasses sequences that include any of the known base analogs of DNA and RNA including, but not limited to, 4-acetylcytosine, 8-hydroxy-N-6-methyladenosine, aziridinylcytosine, pseudoisocytosine, 5-(carboxyhydroxylmethyl)uracil, 5-fluorouracil, 5-bromouracil, 5-carboxymethylaminomethyl-2-thiouracil, 5-carboxymethylaminomethyluracil, dihydrouracil, inosine, N6-isopentenyladenine, 1-methyladenine, 1-methylpseudouracil, 1-methylguanine, 1-methylinosine, 2,2-dimethylguanine, 2-methyladenine, 2-methylguanine, 3-methylcytosine, 5-methylcytosine, N6-methyladenine, 7-methylguanine, 5-methylaminomethyluracil, 5-methoxy-aminomethyl-2-thiouracil, beta-D-mannosylqueosine, 5′-methoxycarbonylmethyluracil, 5-methoxyuracil, 2-methylthio-N-6-isopentenyladenine, uracil-5-oxyacetic acid methylester, uracil-5-oxyacetic acid, oxybutoxosine, pseudouracil, queosine, 2-thiocytosine, 5-methyl-2-thiouracil, 2-thiouracil, 4-thiouracil, 5-methyluracil, N-uracil-5-oxyacetic acid methylester, uracil-5-oxyacetic acid, pseudouracil, queosine, 2-thiocytosine, and 2,6-diaminopurine.

As used herein, the term “sample” is used in its broadest sense. In one sense it can refer to a tissue sample. In another sense, it is meant to include a specimen or culture obtained from any source, as well as biological. In another sense, it is meant to refer to environmental samples. Biological samples may be obtained from animals (including humans) and encompass fluids, solids, tissues, and gases. Biological samples include, but are not limited to bodily fluids such as blood products, such as plasma, serum and the like, urine, semen, saliva, sputum and fractions thereof; proteins, nucleic acids, etc. These examples are not to be construed as limiting the sample types applicable to the present invention. A sample suspected of containing a human chromosome or sequences associated with a human chromosome may comprise a cell, chromosomes isolated from a cell (e.g., a spread of metaphase chromosomes), genomic DNA (in solution or bound to a solid support such as for Southern blot analysis), RNA (in solution or bound to a solid support such as for Northern blot analysis), cDNA (in solution or bound to a solid support) and the like. A sample suspected of containing a protein may comprise a cell, a portion of a tissue, an extract containing one or more proteins and the like.

### DETAILED DESCRIPTION OF THE INVENTION

The present invention relates to lateral flow strip assay system and uses thereof. In particular, the present invention relates to lateral flow assay systems for the simple and inexpensive detection of biomolecules.

Immunochromatographic assays, also known as lateral flow assays, are in vitro diagnostic tests for the detection of a variety of target analytes. The most popular lateral flow based assay is the pregnancy test (e.g., test for human chorionic gonadotropin). Other available tests include tests for monitoring ovulation, detection of infectious disease organisms or cancerous cell markers, analyzing drugs of abuse, and measurement of analytes important to general health. Lateral flow tests are also used in the agriculture, food and environmental sectors.

As the name suggests, this assay utilizes passive flow of fluids in membranes to obtain a result. While they are based on the same immunoassay principles that underlie large clinical analyzers, lateral flow tests are able to generate results without any electromechanical mechanisms or microprocessors. In its most popular format, the test consists of three overlapping membranes that are laid down onto a backing card such that they slightly overlap each other. The first membrane known as the sample pad is usually a glass fiber membrane where the sample is introduced to the test strip. The second membrane is the capture membrane whereupon a capture antibody specific to the target of interest is striped (deposited) onto and is known as the test line. The most popular membrane used as the capture membrane is nitrocellulose, although nylon based membranes or other membranes have been used as well. This membrane is then overlapped by an absorbent pad that acts as a waste reservoir for the excess sample and sustains capillary pressure necessary for the entire sample to be drawn up through the capture membrane.

A conjugate pad is sometimes also used, on to which the conjugate (e.g., antibody coated detector particle) is dried. This conjugate pad is often integrated within the sample pad or found at the interface between the sample pad and the capture membrane whereupon the re-hydration of the conjugate occurs and begins interaction with the targeted analyte. Alternatively, the conjugate can be lyophilized and added to the sample prior to being introduced in the lateral flow test.

To run a test, a fixed volume of sample (e.g., plasma, serum, whole blood, urine or saliva) is added onto the sample pad whereupon certain chemical or biological treatments can occur such as the immobilization of blood cells. The sample then flows into the conjugate pad, re-hydrating the labeled antibody which begins binding to any antigen that may be present in the sample. The sample then flows into the capture membrane by capillary action where the two capture lines are located. The presence of target in the sample leads to formation of a sandwich at the test line that is visible due to the presence of the reporter antibody. Excess conjugate flows to the control line where it leads to the formation of a visible control line. The remaining sample flows into the absorption (waste) pad. For qualitative tests, the development of both lines (test and control lines) signifies a positive test, while the appearance of just the control line is a negative result.

If the control line fails to appear, the result is regarded as invalid. In semi-quantitative and quantitative lateral flow tests, the intensity of the test line is measured using an imaging device (e.g. scanner, camera) and then used to calculate the concentration of target by referring to a standard dilution curve.

There are many parameters that influence the working of a lateral flow test. Such parameters can influence the flow of sample in the test (e.g., membrane pore sizes, detector particle sizes, viscosity of sample, etc) or the binding kinetics of the assay (e.g., affinities and concentrations of the antibodies used). Environmental factors (e.g., temperature, humidity, etc) can also cause variation in test results.

The performance of any diagnostic is usually judged according on its sensitivity and specificity. Sensitivity is the probability of attaining a positive signal given a positive sample, while specificity is the probability of getting a zero signal from a negative sample. By tuning the threshold value (signal above which the sample is positive), assays can be optimized to maximize sensitivity, and specificity. However, it is often difficult to maximize both simultaneously. Depending on the nature of the test, a compromise point is chosen depending on the cost of false negatives vs. false positives.

The sensitivity is dependent upon the Limit of Detection (LOD) of the assay, which is the lowest analyte concentration that can be discerned above background. It is a function of the signal generated from the conjugate, the affinities of the antibodies, background signal of the test and the flow properties of the membrane. It is calculated as three standard deviations of the signal generated from a negative control divided by the slope of the dose response curve. Once the architecture and reagents of the test are chosen, the only way to improve the LOD is by decreasing the background in the test (e.g., as the background signal decreases, its standard deviation also decreases proportionately).

A high signal from a negative control (resulting in a high threshold and hence low sensitivity) can result for a variety of reasons. The causes can be grouped into three principle factors: 1) conjugate related 2) membrane related and 3) sample related which are discussed in turn below.

In the absence of target, the conjugate can bind non-specifically to the test line for a variety of scenarios. Firstly, it can bind as a result of exposed uncovered areas of the particle due to an incomplete coating procedure or loss of coating protein during storage or running of the test. Any unblocked area will tend to bind to protein, in the same way that a conjugate particle gets coated by protein (such as immunoglobulins) during conjugation. The extent of the loss and non-specific binding depends upon the pH, type of particle, strength of antibody conjugation and other physical/chemical variables. Drying of the capture antibody on the test line makes it hydrophobic, which increases the chances of non-specific binding.

Another cause of false negatives is the hydrophobic binding of conjugate clusters to the test line. These clusters can arise due to poor conjugation, or during storage. Such clusters can block the membrane at the point of binding and can cause a restriction of flow, thereby pronouncing the signal from the false positive.

Yet another source of non-specific binding is the conjugate antibody itself. The antibody may denature over time, which increases the probability of it binding to the capture line non-specifically.

Membrane related sources of increased background include non-specific binding to the membrane and inconsistencies in pore sizes that may trap the conjugate as it passes through. These are a function of the manufacturing process of the membrane and can be highly variable even within a single roll of membrane.

Lastly, the sample may introduce substances that increase non-specific binding in the assay. The sample may contain interfering substances that can bridge the conjugate to the capture antibody in the absence of the analyte. Such compounds include, for example, antibodies, hydrophobic proteins and carbohydrates. Human heterophilic antibodies can bind the animal (usually mouse monoclonal) antibodies used in an immunoassay and thus produce spurious results. Cross reactivity due to the presence of fibrin can also lead to increased non-specific signal. The interference from such compounds can be avoided, by adding in compounds to the sample that act as surrogates to bind up the interfering agents within the sample.

In order to afford robustness and consistency to the accuracy of the diagnostic test, such increases in non-specific signal may be minimized. Through the use of well manufactured membranes and the addition of substances to bind the inhibitors in the sample, it is possible to decrease the non-specific binding that arises due to membrane and sample effects.

The use of the lateral flow system has had a long history in the detection and diagnosis of HIV. Developed laboratory technologies rely on the combined and simultaneous detection of the HIV core (p24) protein and HIV-specific antibodies directed against HIV transmembrane proteins. Antibodies against these proteins consistently appear during seroconversion of HIV-infected individuals and remain throughout the course of infection. Such combination immunoassays have targeted reduction of the seronegative window period to decrease the risk of transfusion-transmitted HIV infection. Combining antibody and antigen detection in a single immunoassay format achieves a reduction in the seroconversion window because HIV core protein (p24) appears transiently in the blood and has been used as a marker of antigenemia prior to a detectable humoral immune response to HIV infection.

Accordingly, in some embodiments, the present invention provides compositions, systems and methods for the detection of antigens. The present disclosure utilizes HIV diagnostics to exemplify embodiments of the invention. However, the present invention is not limited to the detection of HIV. The compositions and methods described herein find use in the detection of any suitable protein or antigen.

In some embodiments, the present invention provides infant HIV diagnostics as a self performing point-of-care device. In the first 2 months after birth, HIV positive infants have increasing viral loads but are seropositive due to inheritance of maternal HIV antibodies, making existing tests ineffective. Moreover, HIV negative infants can be seropositive due to the same inheritance of maternal HIV antibodies. Consequently, in some embodiments, detection of HIV in infants utilizes assays targeting the HIV core protein p24 as the principle marker for detection in order to unequivocally verify their true infection state irrespective of the maternal sero-inheritance. In infants, a limit of detection of 0.2-2 picograms of p24 per milliliter of blood is useful in order to identify an HIV positive patient. Thus, in some embodiments, the present invention provides lateral flow systems with sensitivity of at least 100-fold over current technology.

In the conventional immunoassay reaction that is applied to a lateral flow strip, a monoclonal antibody that has been modified to have a chemical tag on it (such as biotin) first reacts and recognizes the p24 protein. Then, a conjugate material that is coated with a second antibody targeted against the p24 protein and is either comprised of a colloidal metal (such as gold or selenium) or a fluorescent microparticle reacts and recognizes a second epitope of the p24 protein (FIG. 4). After this sandwich has been formed, the reaction flows through the nitrocellulose membrane and subsequently passes across a line of capture material (such as neutravidin protein). There, the sandwich particles with the biotin tag are retained and produce a contrasting line, either from a white background to a darker one (as in the case of colloid metals) or from a black background to a brighter one (as in the case of fluorescent dyes).

Embodiments of the present invention provide modified lateral flow assays with modifications to the conjugate used for labeling and the configuration of the test strip to allow for efficient capture of the sandwich particles. Experiments conducted during the course of development of embodiments of the present invention demonstrated that the positioning of the neutravidin test-line provides a benefit in maximizing particle capture while minimizing background noise on the membrane as well as non-specific signal at the test line.

In some embodiments, latex microspheres containing organic dye that fluoresces brightly are utilized. In some embodiments, spheres are obtained from a commercial vendor (e.g., Sphereotech; Lake Forest, Ill.). In some embodiments, spheres comprise saturating amounts of fluorescent organic dye. In some embodiments, particles are approximately 300 nm in diameter, although other sizes may be utilized. Experiments conducted during the course of development of embodiments of the present invention demonstrated that in order to favor kinetics of the reaction and facilitate consistent flow through the nitrocellulose membrane, a particle size of 300 nm or larger is preferred.

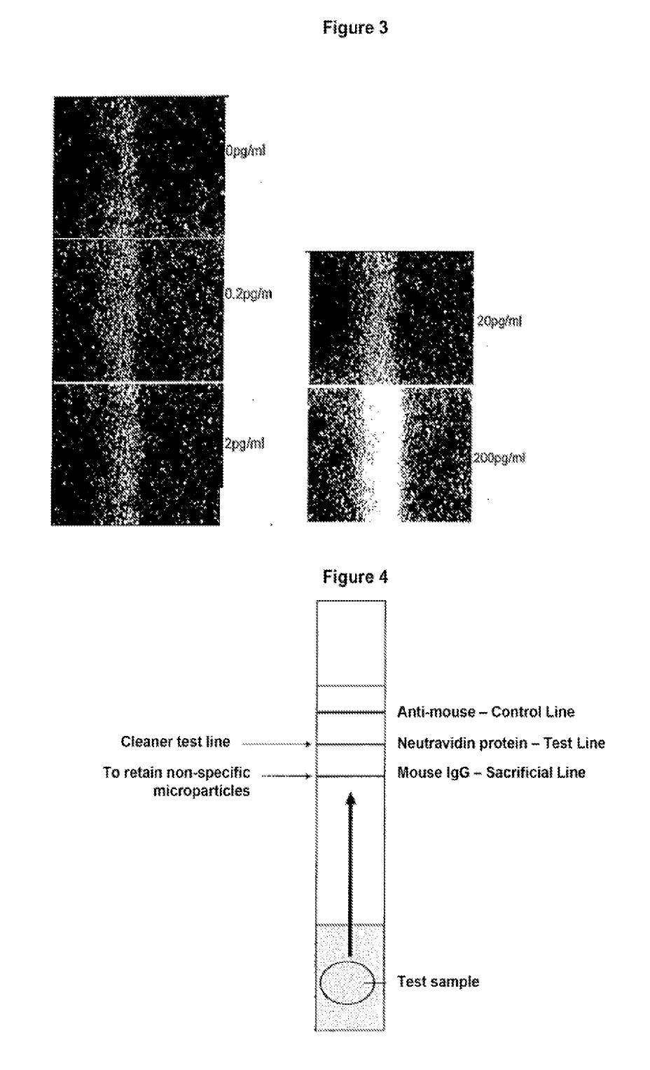

Experiments conducted during the course of development of embodiments of the present invention demonstrated that the modified assays described herein were able to successfully detect p24 antigen levels as low as 2 picograms per milliliter in a diluted human plasma matrix (FIG. 3).

In some embodiments, lateral flow assays systems are further modified to reduce non-specific signal. The present invention is not limited to a particular mechanism. Indeed, an understanding of the mechanism is not necessary to practice the present invention. Nonetheless, it is contemplated that one reason for non-specific signal at the test line is heterophile antibody bridging interactions between the unbound biotinylated antibodies. It is also contemplated that the fluorescent microspheres coated with the second monoclonal antibody as well as fluorescent latex particles coated with the mouse antibody contain particles that are inherently sticky because when they are coated with antibody and some of the antibody is denatured and becomes sticky causing the particles to bind non-specificly to the membrane, especially in areas coated with protein, such as the test line where the membrane flow is slower because of reduced pore size with the bound protein and denatured protein from the binding process.

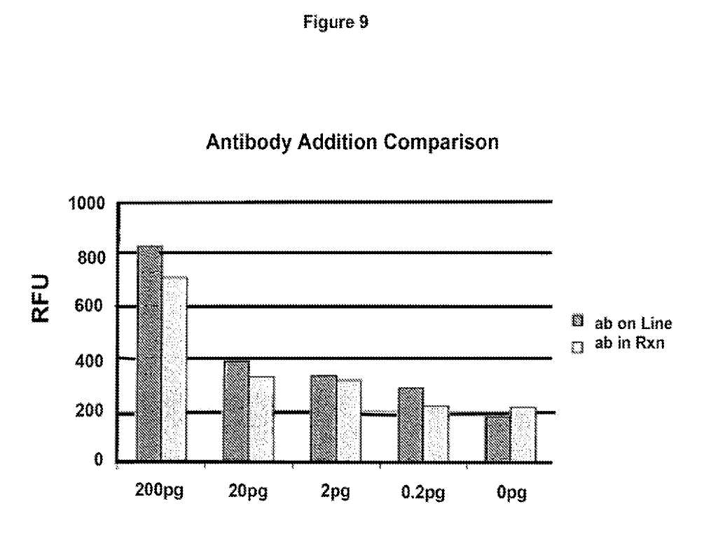

Accordingly, in some embodiments, the interfering interactions are reduced by introducing a ‘sacrificial’ line on the nitrocellulose membrane comprised of either immunoglobulin molecules or their sub-components or adding heterophile blockers in the reaction mix (FIG. 9). In some embodiments, the sacrificial line filters out the improperly functionalized conjugate and the big conjugate clusters by allowing them to bind. Its composition can vary, but in some embodiments, it is composed of a compound similar to that used in the test line.

Whole IgG molecules from different species as well as just the Fc and Fab1Fab″ components of the immunoglobulins jetted directly on the nitrocellulose, slightly upstream of the test line were assayed. The most effective immunoglobulin was that of mice although immunoglobulins from other species had the same effect and are suitable for use in such embodiments. In some embodiments, the sacrificial line is positioned within close proximity (2-4 mm) of the test line.

Experimentation conducted during the course of development of embodiments of the present invention revealed that the nature of the interaction between components of the diagnostic chemistry with sacrificial test line is due to the presence of mouse antibody coating the fluorescent latex microparticle. For instance, uncoated latex microparticles do not react with the sacrificial line, whereas particles coated with mouse monoclonal antibody as well as those that have formed a complete sandwich (antibody #1 bound to antigen bound to biotinylated antibody #2) bind with equal efficacy to the sacrificial test line (FIG. 6).

Enhancement of signal-to-noise ratios was not obtained when whole IgG molecules or the Fc and Fab/Fab″ components of the immunoglobulins were mixed in the test reaction as compared to interacting with identical material jetted directly on the nitrocellulose (FIG. 6).

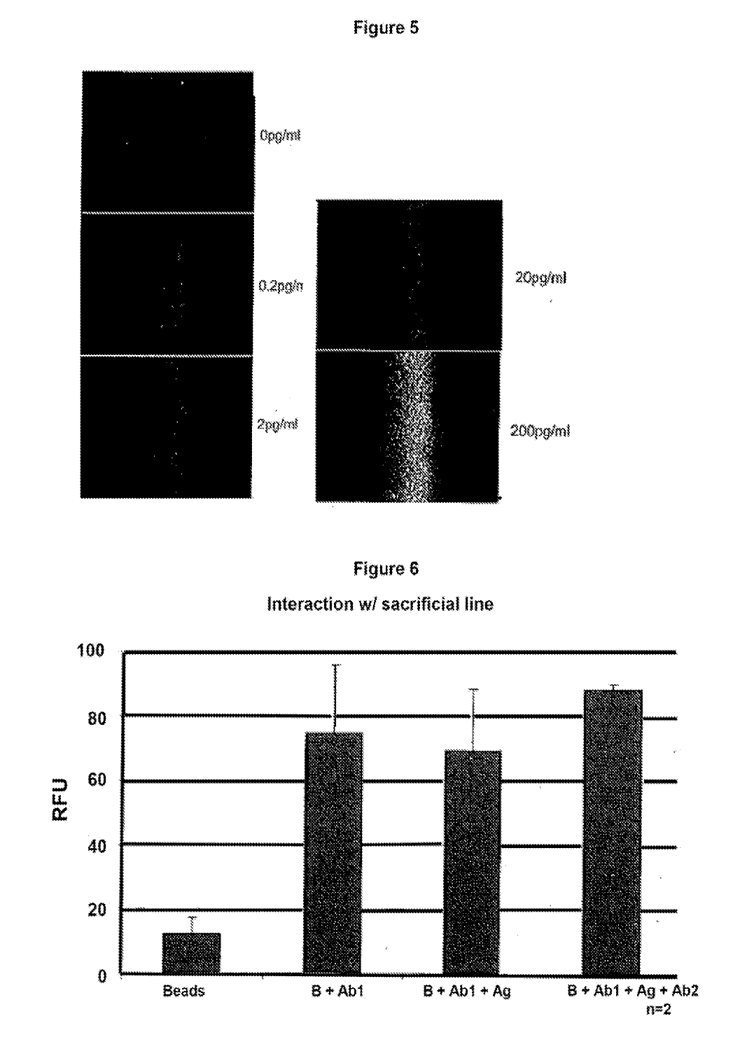

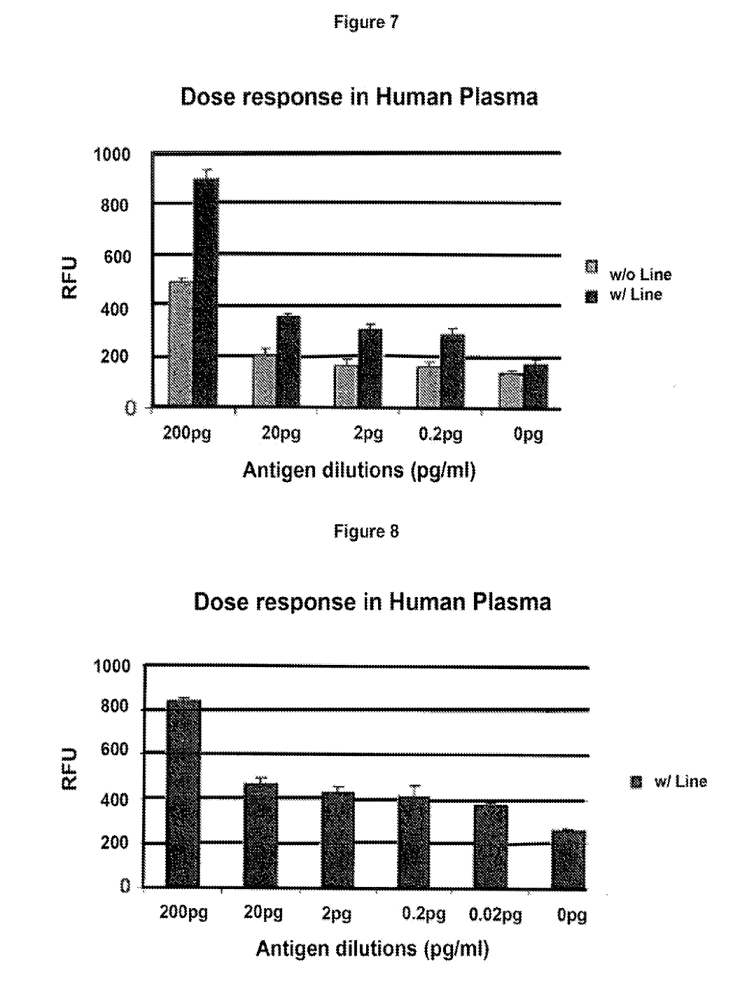

With the introduction of the sacrificial test line, our analytical limit of detection improved from 2 to 0.02 picograms of p24 protein per milliliter (FIGS. 5, 7 and 8) when the reaction was run in either human plasma or biological buffer containing equivalent concentrations of protein. This 100-fold gain of sensitivity was afforded through two independent means of signal-to-noise enhancement. For example, the net test line signals were calculated by subtracting the background fluorescence from the signal, the greatest gain in contrast was through reduction of background noise in the nitrocellulose membrane. Secondly, non-specific binding occurring at the test line was reduced. With these two improvements, discrimination of sub-picrogram levels of analyte was clearly discernible.

Embodiments of the present invention described herein, which costs approximately 25 cents per test, are comparable to performance achieved formally with high-end instrumentation and biological tests such as the ELISA assay.

The assay systems described herein find use in a variety of immunoassay applications. Examples include, but are not limited to, low-analyte infectious diseases or markers for environmental monitoring.

In some embodiments, the analyte to be detected is a protein, peptide, small molecule; antibody, nucleic acid, virus, virus particle, drug, drug metabolite or small molecule. Specific examples include, but are not limited to, human chorionic gonadotrophin, luteinizing hormone, estrone-3-glucoronide, pregnanedio13-glucoronide, insulin, glucagon, relaxin, thyrotropin, somatotropin, gonadotropin, follicle-stimulating hormone, gastrin, bradykinin, vasopressin, polysaccharides, estrone, estradiol, cortisol, testosterone, progesterone, chenodeoxycholic acid, digoxin, cholic acid, digitoxin, deoxycholic acid, lithocholic acids; vitamins, thyroxine, triiodothyronine, histamine, serotorin, prostaglandin, drugs, drug metabolites, ferritin or CEA.

In some embodiments, immunoassays utilize antibodies to a purified protein (e.g., analyte). Such antibodies may be polyclonal or monoclonal, chimeric, humanized, single chain or Fab fragments, which may be labeled or unlabeled, all of which may be produced by using well known procedures and standard laboratory practices. See, e.g., Burns, ed., Immunochemical Protocols, 3^{rd} ed., Humana Press (2005); Harlow and Lane, Antibodies: A Laboratory Manual, Cold Spring Harbor Laboratory (1988); Kozbor et al., Immunology Today 4: 72 (1983); Köhler and Milstein, Nature 256: 495 (1975). In some embodiments, commercially available antibodies are utilized.

The devices and methods of the present invention are suitable for use with a variety of sample types. Exemplary sample types include, but are not limited to, blood, serum, nasal fluid, urine, sweat, plasma, semen, cerebrospinal fluid, tears, pus, amniotic fluid, saliva, lung aspirate, gastrointestinal contents, vaginal discharge, urethral discharge, chorionic villi specimens, skin epithelials, genitalia epithelials, gum epithelials, throat epithelials, hair or sputum.

In some embodiments, kits, systems and/or devices of the present invention are shipped containing all components necessary, sufficient or useful to perform immunoassays. In other embodiments, additional reaction components are supplied in separate vessels packaged together into a kit.

Any of these compositions, alone or in combination with other compositions disclosed herein or well known in the art, may be provided in the form of a kit. Kits may further comprise appropriate controls and/or detection reagents. Any one or more reagents that find use in any of the methods described herein may be provided in the kit.

All publications, patents, patent applications and sequences identified by accession numbers mentioned in the above specification are herein incorporated by reference in their entirety. Although the invention has been described in connection with specific embodiments, it should be understood that the invention as claimed should not be unduly limited to such specific embodiments. Modifications and variations of the described compositions and methods of the invention that do not significantly change the functional features of the compositions and methods described herein are intended to be within the scope of the following claims.

We claim:

1. An assay device for the detection of the presence or absence of an analyte in a sample, comprising:
(a) A sample receiving membrane which conducts flow of a sample and is in flow contact with:
(b) An analyte detection membrane which conducts flow of the sample, comprising one or more of i) a labeling reagent absorption zone comprising a labeling reagent, ii) an analyte-reagent complex capture zone comprising an analyte capture reagent, iii) a control reagent capture zone comprising control reagent; and a sacrificial zone comprising non-specific binders,
wherein said labeling reagent is capable of forming a complex with an analyte to form an analyte-labeling reagent complex, said analyte capture reagent is capable of binding said analyte-labeling reagent complex, said control reagent is capable of binding said labeling reagent, and said non-specific binders bind to unbound analyte specific antibodies or other analyte specific components in said sample.

2. The assay device of claim 1, wherein the flow contact between the sample receiving membrane and analyte detection membrane is lateral flow contact.

3. The assay device of claim 1, wherein the labeling reagent comprises an antibody specific for said analyte.

4. The assay device of claim 3, wherein said antibody is selected from the group consisting of an IgG antibody, an IgM antibody, an IgA antibody and a portion thereof.

5. The assay device of claim 4, wherein the antibody or portion thereof is selected from the group consisting of mouse, goat, sheep, rat, rabbit, cow, human and chimeras thereof.

6. The assay device of claim 1, wherein the sample receiving membrane and the analyte detection membrane are enclosed in a housing.

7. The assay device of claim 6, wherein said housing comprises a sample application aperture and an observation window positioned to display the labeling reagent capture zone, a detection zone and said control zone.

8. The assay device of claim 1, further comprising an absorbent sink in lateral flow contact with said analyte detection membrane.

9. The assay device of claim 1, wherein said sacrificial zone is located approximately 14 mm from the distal end of said sample receiving membrane and said analyte-reagent complex capture zone is located approximately 16 mm from the distal end of said sample receiving membrane.

10. The assay device of claim 9, wherein said analyte capture reagent comprises a label.

11. The assay device of claim 10, wherein said label is a fluorescent label.

12. The assay device of claim 12, wherein said fluorescent label is contained in a microsphere.

13. The assay device of claim 1, wherein said binders are immunoglobulins.

14. A method of detecting the presence of an analyte in a sample comprising:
I) applying a sample to an assay device; wherein said assay device comprises (a) A sample receiving membrane which conducts flow of a sample and is in flow contact with:
(b) An analyte detection membrane which conducts flow of the sample, comprising i) a labeling reagent absorption zone comprising a labeling reagent, ii) an analyte-reagent complex capture zone comprising an analyte capture reagent, iii) a control reagent capture zone; and a sacrificial zone comprising non-specific binders,
wherein said sample flows from said sample receiving membrane to said analyte detection membrane under conditions such that said labeling reagent forms a complex with said analyte to form an analyte-labeling reagent complex and said analyte capture reagent binds to said analyte-labeling reagent complex; and
II) detecting the presence of said analyte.

15. The method of claim 14, wherein said analyte is selected from the group consisting of a protein, peptide, small molecule; antibody, nucleic acid, virus, virus particle, drug, drug metabolite and small molecule.

16. The method of claim 15, wherein said analyte is selected from the group consisting of human chorionic gonadotrophin, luteinizing hormone, estrone-3-glucoronide, pregnanedio13-glucoronide, insulin, glucagon, relaxin, thyrotropin, somatotropin, gonadotropin, follicle-stimulating hormone, gastrin, bradykinin, vasopressin, polysaccharides, estrone, estradiol, cortisol, testosterone, progesterone, chenodeoxycholic acid, digoxin, cholic acid, digitoxin, deoxycholic acid, lithocholic acids; vitamins, thyroxine, triiodothyronine, histamine, serotorin, prostaglandin, drugs, drug metabolites, ferritin and CEA.

17. The method of claim 14, wherein the sample is selected from the group consisting of blood, serum, nasal fluid, urine, sweat, plasma, semen, cerebrospinal fluid, tears, pus, amniotic fluid, saliva, lung aspirate, gastrointestinal contents, vaginal discharge, urethral discharge, chorionic villi specimens, skin epithelials, genitalia epithelials, gum epithelials, throat epithelials, hair and sputum.

18. An kit for the detection of the presence or absence of an analyte in a sample, comprising:
(a) A sample receiving membrane which conducts flow of a sample and is in flow contact with:
(b) An analyte detection membrane which conducts flow of the sample, comprising i) a labeling reagent absorption zone comprising a labeling reagent, ii) an analyte-reagent complex capture zone comprising an analyte capture reagent, iii) a control reagent capture zone comprising control reagent; and a sacrificial zone comprising non-specific immunoglobulins,
wherein said labeling reagent is capable of forming a complex with an analyte to form an analyte-labeling reagent complex, said analyte capture reagent is capable of binding said analyte-labeling reagent complex, said control reagent is capable of binding said labeling reagent, and said non-specific immunoglobulins bind to unbound analyte specific antibodies.
